(19) United States
(12) Patent Application Publication
(10) Pub. No.: US 2010/0080768 A1
(43) Pub. Date: Apr. 1, 2010

(54) Compositions and Methods for the Treatment of Inflammatory Dermatosis and Other Pathological Conditions of the Skin

(76) Inventors: Thomas L. McGraw, Pompano Beach, FL (US); Donald M. McGraw, Pompano Beach, FL (US); Keli C. McGraw, Pompano Beach, FL (US); Donald V. McGraw, Red Oak, TX (US)

Correspondence Address: MAYBACK & HOFFMAN, P.A., 5722 S. FLAMINGO ROAD #232, FORT LAUDERDALE, FL, 33330, US

(21) Appl. No.: 12/238,481

(22) Filed: Sep. 26, 2008

(51) Int. Cl. A61K 31/765; A61P 29/00

(52) U.S. Cl. 424/78.37

(57) ABSTRACT

The present invention relates to a composition used as a vehicle for percutaneous absorption of Pharmaceutical and Cosmaceutical active agents that comprises Dimethiconol (hydroxyl-terminated polydimethydimethylsiloxane), dimethicone-350 (polydimethylsiloxane-350), cyclomethicone-5 nf (decamethylcyclopentasiloxane), alkymeth siloxane copolyol-lauryl peg/ppg 18/18 methicone (alkymethyl siloxane copolyol), cyclopentasiloxane and dimethicone Crosspolymer (silicone elastomer and decamethylcyclopentasiloxane), stearoxytrimethylsilane and stearyl alcohol (silicone wax), and deionized water. This composition serves several key applications: (1) it is a vehicle for percutaneous absorption of Pharmaceutical and Cosmaceutical active agents; (2) it acts as a method for utilizing other compositions in the treatment of inflammatory conditions of the skin including, but by no means limited to, atopic dermatitis (eczema), allergic contact dermatitis, seborrheic dermatitits, psoriasis, xerosis and atopia; (3) it is a treatment of inflammatory conditions of mucosae; (4) it relates to other compositions and methods for protecting and enhancing the barrier function of the skin.

### BACKGROUND OF THE INVENTION

1. Field of the Invention

The present invention relates to a composition used as a vehicle for the percutaneous absorption of pharmaceutical and cosmaceutical active agents that includes dimethiconol (hydroxyl-terminated polydimethylsiloxane), dimethicone-350 (polydimethylsiloxane-350), cyclomethicone-5 nf (decamethylcyclopentasiloxane), alkymeth siloxane copolyol-lauryl peg/ppg 18/18 methicone (alkymethyl siloxane copolyol), cyclopentasiloxane and dimethicone Crosspolymer (silicone elastomer and decamethylcyclopentasiloxane), stearoxytrimethylsilane and stearyl alcohol (silicone wax), and deionized water. The composition serves, among others, the following applications: (1) it is a vehicle for percutaneous absorption of Pharmaceutical and Cosmaceutical active agents; (2) it acts as a method for utilizing other compositions in the treatment of inflammatory conditions of the skin including, but not limited to, atopic dermatitis (eczema), allergic contact dermatitis, seborrheic dermatitits, psoriasis, xerosis, and atopia; (3) it is a treatment of inflammatory conditions of mucosae; and (4) it relates to other compositions and methods for protecting and enhancing the barrier function of the skin.

2. Description of the Related Art

The stratum corneum is the outer-most layer of the skin that functions as a barrier against chemicals and other stress agents found in the environment and is responsible for the regulation of the water content of the skin. The complex arrangement of lipids in the intercellular space of the stratum corneum is responsible for the establishment of this normal barrier function. Multi-layered structures of cholesterol, ceramides, fatty acids, and some other minor lipids provide a barrier to the transport of substances into or through the skin with the overall structure of the stratum corneum acting as the frontline barrier to the skin. The link between skin barrier function and skin health is apparent from the inflammation of the skin when lipids are extracted. Put differently, when the skin's barrier function is impaired, the other layers of the skin can be injured and have a response to that injury in the form of inflammation. The mechanism to repair a skin barrier suffering from such damage can be threefold. It can involve: (1) proper lipid selection; (2) semi-occlusion film formation of the skin tissue during the repair phase; and (3) the replacement of water and ion balance in the tissue to create barrier homeostasis.

Inter-and Intra-Individual Differences in Human Stratum Corneum Lipid Content Related to Physical Parameters of Skin Barrier Function In Vivo. J Invest Dermatol, 1999 112;72-77;doi:10.1046/j.-1523-1747.1999.00481.x, Norlen, et al. concluded that lipid organizations have been found in molar concentration of 32:37:16:15 (cholesterol:ceramides:free fatty acids:cholesterol esters) with both the stratum corneum lipids (cholesterol, ceramides, and free fatty acids) and minor fractions (e.g., cholesterol sulfate, etc.) possibly affecting stratum corneum lipid organization. In diseased skin, the barrier function is frequently impaired due to a deviation from this lipid concentration. As disclosed in U.S. Pat. No 5,643,899 issued to Elias et al on Jul. 1, 1997, application of a lipid-containing composition to the skin is a known approach for enhancing the barrier function of the stratum corneum. For some time, those skilled in the art have believed that it is necessary to apply this molar concentration of about 32:37:16:15 of lipid components to the stratum corneum in order to replenish and repair the barrier function of the skin. Recent metabolic studies have also demonstrated the importance of both cholesterol and fatty acids to barrier homeostasis. Deficiencies or other irregularities in these substances often translate into adverse health effects in the skin, a point best exemplified in the context of linoleic acid deficiencies. Linoleic acid is known to be required for cutaneous barrier function, the majority of which is esterfied into ceramide. Deficiencies in linoleic acid content in essential fatty acids lead to the development of an abnormal permeability barrier. Such deficiencies and the subsequently developing abnormal permeability barrier have been attributed to the substitution of oleic acid for linoleic acid in the epidermal spingolipids.

The stratum corneum possesses approximately 30% water (which is associated with its elasticity), ten percent of which is bound to lipids with the remaining 20% (which is resistant to solvent and water extraction) possibly secondary to keratin components. Such water content is vital to processes such as plasticization of the stratum corneum which is regarded as a function dependent on water content. For a stratum corneum to be healthy, it must be able to maintain an adequate level of water against the evaporative diffusion gradient created by the presence of low relative humidity. This fact is more apparent when one considers the structure of the stratum corneum. The innermost layers of the stratum corneum contain a high level of water while the outermost layers contain a water level dependent on the ambient relative humidity. Thus, a healthy stratum corneum should contain about 10% tightly held water.

Disease State

Atopic dermatitis is a chronic disease that affects skin. The word “dermatitis” means inflammation of the skin and “atopic” refers to a group of diseases where there is often an inherited tendency to develop other allergic conditions. Atopic dermatitis is often referred to as eczema, the most common of many types of atopic dermatitis. With atopic dermatitis, the skin becomes extremely itchy, often causing the person to scratch, which leads to redness, swelling, cracking, weeping, clear fluids, crusting, and scaling. In most cases, there are changes in the disease state, ranging from periods of time when the disease is intense (called exacerbations or flares) followed by periods when the inflammation resulting from such exacerbations improves or disappears. However, the skin of those suffering from atopic dermatitis often remains dry and easily irritated and continues to pose a problem for many throughout their lives.

Atopic dermatitis is extremely common, affecting both males and females and accounting for 10% to 20% of all visits to dermatologists. Scientists estimate that as much as 65% of atopic dermatitis sufferers develop symptoms in the first year of life and 90% develop symptoms before the age of five. According to the National Institute of Health, an estimated 20% of infants and young children experience symptoms of the disease with roughly 60% of these infants continuing to have one or more symptoms of atopic dermatitis into adulthood. These statistics translate into more than fifteen million people in the United States having symptoms of the disease.

In addition, atopic dermatitis is a common cause of workplace disability as demonstrated by the National Institute of Health in a recent analysis of the health insurance records of five million Americans under the age of 65. In this analysis, medical researchers found that approximately two and one-half percent of the records surveyed demonstrated an infliction of atopic dermatitis. The annual insurance payments for the medical care of atopic dermatitis ranged from $580 to $1,250 per patient with more than one-quarter of each patient's total health care cost attributable to atopic dermatitis and related conditions. Moreover, the researchers projected that United States health insurance companies spend more than one billion dollars per year on atopic dermatitis.

Common skin features of atopic dermatitis include: Atopic pleat (an extra fold of skin that develops under the eye); Cheilitis (inflammation of the skin on or around the lips); Hyperlinear palms (an increased number of skin creases on the palms); hyperpigmented eyelids (eyelids that have become darker in color from inflammation or hay fever); Ichthyosis (dry, rectangular scales on the skin); Keratosis pilaris (small, rough bumps, generally on the face, upper arms, and thighs); Lichenification (thick, leathery skin resulting from constant scratching and rubbing); Papules (small raised bumps that may open when scratched and become crusty and infected); and Urticaria (hives, which are red, raised bumps that often occur after exposure to an allergen at the beginning of flares or after exercise or a hot bath).

According to the National Institute of Health, researchers have noted differences in the skin of people with atopic dermatitis that may contribute to the symptoms of the disease. The outer layer of skin, called the epidermis, is divided into two parts: an inner part containing moist, living cells, and an outer part, known as the horny layer or stratum corneum, containing dry, flattened, dead cells. Under normal conditions the stratum corneum acts as a barrier, keeping the rest of the skin from drying out and protecting other layers of the skin from damage caused by irritants and infections. When this barrier is damaged irritants act more intensely on the skin. The skin of a person with atopic dermatitis loses moisture from the epidermal layer, allowing the skin to become very dry and reducing its protective abilities. Thus, when combined with an abnormal skin immune system, a person's skin is more likely to become infected by bacteria and viruses.

Therefore, the main goals in the treatment of atopic dermatitis are healing the skin, restoring the barrier function of the skin, and preventing flares. Current treatments used to achieve these goals include the use of immuno-modulators, coal tar, keratolytics, anthralin, topical vitamin D-3 analogues, and tazarotene with corticosteroids. Unfortunately, these products are associated with side effects, such as irritation and toxicity, in addition to presenting the added problem of non-compliance of the dosage regiment due to their poor aesthetic properties.

Each of the above listed treatment methods presents disadvantages. For example, tar preparation produces the disadvantages of irritation, staining, and a foul odor. With immuno-suppressives, the Food and Drug Administration (FDA) limits the use of pimecrolimus or tacrolimus ointment because of potential cancer risk. Moreover, the FDA also stresses that these medications should only be used only as directed and after trying all other options. Further, these calcineurin inhibitors are not approved for children younger than two years of age.

The Composition Excipients

In U.S. patent application Ser. No. 09/379,928, various compositions for improving skin health are described. The compositions claimed to have been found to provide benefits for skin health were described as containing a variety of potential components and, in some forms, the compositions included natural fats and oils, sterols, sterol derivatives, humectants, and surfactants. These compositions have been found to improve skin health even though they do not necessarily include any ceramides. Though the exact mechanism of functionality was not known, one hypothesis was that an emulsion of the lipid components (natural fats and sterols) was formed in the humectant component. The ointment formulation, which could include petrolatum, would form an occlusive film on the skin that would trap water between the skin and the occlusive film. This trapped water was predicted to facilitate uptake of the emulsion and therefore the natural oils and sterols by the skin.

Therefore a need exists to overcome the problems with the prior art as discussed above.

### SUMMARY OF THE INVENTION

Briefly, in accordance with one embodiment of the present invention, disclosed is a composition suitable for topical administration and a method for the treatment of inflammatory dermatosis and other pathological conditions of the skin. The method entails topical administration of the inventive composition to the site of a dermatological disease or disorder. This topically-administered composition includes: dimethiconol(hydroxyl-terminated polydimethylsiloxane), dimethicone-350 (polydimethylsiloxane-350), cyclomethicone-5 nf (decamethylcyclopentasiloxane), alkymeth siloxane copolyol-lauryl peg/ppg-18/18 methicone (alkymethyl siloxane copolyol), cyclopentasiloxane and dimethicone Crosspolymer (silicone elastomer and decamethylcyclopentasiloxane), stearoxytrimethylsilane and stearyl alcohol (silicone wax), Alpha bisabolol, glycyrrhizinic acid, liposomal with vitamins A, E, C, cholesterol sulfate, caprylic/capric triglyceride, panthenol, glycerin, and sodium hyaluronate.

The present invention also relates to a method whereby the composition is provided as a transdermal base used for the percutaneous absorbption of pharmaceutical and cosmaceutical active agents including, but not limited to: antiboditics, topical steroids, topical corticosteroid, coal tar, folic acid antagonists (such as methotrexate), antibiotics (such as erythromycin), antiemetic (such as scopolamine), a prostaglandin (such as misoprostol), an anti-inflammatory (such as ibuprofen, ketoprofen or diclofenac), biologically active proteins (such as phytospingosine or spingosine), analgesic (such as ketamine), a hormone (such as progesterone or testosterone), a corticosteroid (such as clobesterol), a vasodilator (such as isosorbide dinitrate), or other pharmaceutical agents.

In certain embodiments, the composition of the present invention is suitable for topical administration in the form of oil/water emulsion, water/oil emulsion, water/oil/water emulsion, silicone/water emulsion, water/silicone emulsion, water/wax emulsion, oil/water/silicone emulsion, hydro-alcohol gel, cream, or an ointment. In some embodiments, the composition of the present invention can be used by itself or in admixture with one or more medicaments or excipients (preferably film forming components which adhere to the epithelium).

In another embodiment, the present invention relates to a method of topically administering to the site of a skin condition, a composition containing sufficient amounts of agents to result in the prevention, recurrence, or onset of a skin condition.

In another embodiment, the present invention relates to a method of treating, preventing or restoring the skin's barrier function.

In another embodiment, the present invention relates to a method of topically administering to the site of a skin condition a composition that includes:
Phase A: Dimethiconol (hydroxyl-terminated polydimethylsiloxane) (2.0% w/w), dimethicone-350 (Polydimethylsiloxane-350) (5.0% w/w);
Phase B: Cyclomethicone-5 nf (decamethylcyclopentasiloxane) (15.0% w/w);
Phase C: Alkymeth Siloxane copolyol-lauryl peg/ppg 18/18 methicone (alkymethyl Siloxane copolyol) (2.0% w/w);
Phase D: Cyclopentasiloxane and Dimethicone Crosspolymer (silicone elastomer and decamethylcyclopentasiloxane) (10.0% w/w), Stearoxytri methyl si lane and stearyl alcohol (silicone wax) (5.0% w/w);
Phase E: Sodium polyacrylate, dimethicone, cyclopentasiloxane, trideceth-6, and peg/ppg-18/18 dimethicone (sodium polyacrylate in dimethicone) (5.0% w/w); and
Phase F: Deionizer water (68.0% w/w) and preservative.

With the preparation of such claimed composition entailing: (1) mixing ingredients of phase A together; (2) mixing ingredients of phase B together; (3) mixing ingredients of phase C together; (4) heating phase D to 50 degrees Celcius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; (8) and finally the continuation of mixing for 15 minutes and then homogenization of the emulsion.

Optionally, in another embodiment, the present invention relates to a method for treating hypertrophic scars by applying to scar tissue a composition that includes dimethiconol (hydroxyl-terminated polydimethylsiloxane), dimethicone-350 (polydimethylsiloxane-350), cyclomethicone-5 nf (decamethylcyclopentasiloxane), alkymeth siloxane copolyol-lauryl peg/ppg 18/18 methicone (alkymethyl siloxane copolyol) (2% w/w), cyclopentasiloxane and dimethicone Crosspolymer (silicone elastomer and decamethylcyclopentasiloxane), stearoxytrimethylsilane and stearyl alcohol (silicone wax). This method is effective in reducing the size and appearance of scars.

In another embodiment, the present invention relates to a method of managing and relieving the burning experienced with various types of dermatoses, including but by no means limited to atopic dermatitis, allergic contact dermatitis, and radiation dermatitis. This method entails topically administering to the site of the dermatitis a composition comprised of dimethiconol(hydroxyl-terminated polydimethylsiloxane), dimethicone-350 (polydimethylsiloxane-350), cyclomethicone-5 nf (decamethylcyclopentasiloxane), alkymeth siloxane copolyol-lauryl peg/ppg 18/18 methicone (alkymethyl siloxane copolyol) (2.0% w/w ), cyclopentasiloxane and dimethicone Crosspolymer (silicone elastomer and decamethylcyclopentasiloxane), stearoxytrimethylsilane and stearyl alcohol (silicone wax), Alpha bisabolol, glycyrrhizinic acid, liposomal with A, E, C, cholesterol, panthenol, shea blend emulsion, sulfate, caprylic/capric triglyceride, glycerin, and sodium hyaluronate. More particularly stated, the composition of the present invention relates to a method of treating an inflammatory condition. Such method entails topically administering to the inflamed site the claimed composition containing actives in an amount effective to treat or prevent the condition causing such inflammation.

In another embodiment, the present invention relates to a method of managing and relieving mucosal inflammatory conditions and neuropathic pain (particularly of the mouth and gingival).

In a another embodiment, the present invention relates to a method of treating or preventing inflammatory conditions selected from the group consisting of dermatitis conditions and skin impairments.

### DETAILED DESCRIPTION

While the specification concludes with claims defining the features of the invention that are regarded as novel, it is believed that the invention will be better understood from a consideration of the following description. It is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting; but rather, to provide an understandable description of the invention.

The present invention, according to one embodiment, relates to a composition suitable for topical administration which comprises:
Phase A: Dimethiconol (hydroxyl-terminated polydimethylsiloxane) (1.0-35.0% w/w), dimethicone-350 (Polydimethylsiloxane-350) (1.0-20.0% w/w);
Phase B: Cyclomethicone-5 nf (decamethylcyclopentasiloxane) (1.0-40.0% w/w);
Phase C: Alkymeth Siloxane copolyol-lauryl peg/ppg 18/18 methicone (alkymethyl Siloxane copolyol) (1.0-5.0% w/w);
Phase D: Cyclopentasiloxane and Dimethicone Crosspolymer (silicone elastomer and decamethylcyclopentasiloxane) (1.0-75.0% w/w), Stearoxytrimethylsilane and stearyl alcohol (silicone wax ) (1.0-35.0%));
Phase E: sodium polyacrylate and dimethicone and cyclopentasiloxane and trideceth-6 and peg/ppg-18/18 dimethicone (sodium polyacrylate in dimethicone) (1.0-10.0% w/w); and
Phase F: Deionizer water (0.1-70.0% vv/w).

The method of preparation for the composition described includes the steps of: (1) mixing ingredients of phase A together; (2) mixing ingredients of phase B together; (3) mixing ingredients of phase C together; (4) heating phase D to 50 degrees Celsius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; (8) continuing mixing for 15 minutes and then homogenizing the emulsion.

In certain embodiments, the claimed composition is in the form of oil/water emulsion, water/oil emulsion, water/oil/water emulsion, silicone/water emulsion, water/silicone emulsion, water/wax emulsion, oil/water/silicone emulsion, hydro alcohol gel, cream, or ointment.

Compositions according to embodiments of the present invention may further contain peg-12 dimethicone as an emulsifier for various oils and active ingredients. This emulsifier is capable of forming silicone vesicles which provide the aesthetic benefits of silicones in addition to acting as an effective delivery system. The concentration of usage of peg-12 dimethicone is 4.0% w/w. Other excipients that can be included in the compositions of the present invention include, but are not limited to, co-emulsifiers, preservatives, or viscosity increasing agents. Preferred examples of such excipients include, but are by no means limited to Lauryl peg/ppg-18/18 methicone (a co-emulsifier), Kathon CG and Germaben II (preservatives), and Lauryl peg/ppg-18/18 Methicone (a viscosity additive).

The composition of the present invention may further contain one or more skin conditioning agents, which are materials that soften, smooth, supple, coat, lubricate, reduce flaking, moisturize, and/or protect the skin against wetness or irritation. Thus, skin conditioning agents may maintain the normal healthy skin condition. For example, one type of skin conditioning agent, emollients, are particularly useful in improving the dry skin condition by restoring moisture levels. In addition to restoring moisture levels, emollients add to the softness, smoothness, pliability, and flexibility of the skin. Another type of skin conditioning agent, generally referred to as barrier protectants, form an occlusive layer on the skin surface that prevents or retards moisture loss from the deeper layers of the skin to the atmosphere. These occlusive agents also provide barrier protection to the skin as a defense against irritants. Skin conditioning agents will generally comprise from about 1 to 20 weight percent (% w/w) of the composition, with a preferable range from about 3 to 5 weight percentage (% w/w) of the composition. Exemplary skin conditioning agents useful in the present invention include, but are not limited to, fatty acid esters, polysiloxanes, silicone waxes, fluids and gums, fatty alcohol esters, polyhydroxy alcohols, sterols, sterol esters, sphingolipids, and phospholipids. Preferable polysiloxanes, silicone waxes, fluids and gums, or fatty acid ester skin conditioning agents include co-emulsifier isopropyl myristate and co-emulsifier isopropyl palmitate. Also useful in the present invention are esters of polyhydroxy alcohols which are generally used as the humectants-type skin conditioning agent. This type of ester may include, but is not limited to glycerin, glycerol ester (including glycerides), or glycolic esters which are derived from glycols, propylene glycol, or polyethylene glycols. Also useful in the present invention as skin conditioning agents are sphingolipids such as ceramides, sphingosines, phytospingosines, and phospholipids (such as lecithin, phosphatidylinositol, or sphingomyelin).

In addition to the ingredients hereto mentioned, the topical composition of the present invention will include one or more film forming ingredients. Effective skin\-conditioning agents with superior barrier properties consist of a mixture of components that stimulate the skin's water-barrier, forming lipid complex. Such components include, but are not limited to, sterols, sterol esters, triglycerides, hydrolyzed protein, collagen derived protein, acetamide-MEA, and/or polysiloxanes.

The compositions of the invention can be used by themselves or in admixture with one or more medicaments or excipients. It is preferable that such medicaments or excipients are film-forming components which adhere the epithelium. This may include, but is not limed to, water, surfactants, skin care ingredients, anti-oxidants, pH buffering systems, antibacterial, antiviral, antifungal, analgesic, local anesthetics, and pharmaceutical actives. Most of these materials are well known in the art as additives for such formulations.

According to a preferred embodiment, the compositions of the invention will further contain one or more compounds which are safe and effective skin-care ingredients. Such ingredients may be incorporated in the present composition in an amount ranging from about 0.01 to 15%, with a preferable range from 0.1 to 10%. Such materials include the F.D.A.'s generally recognized as safe and effective (GRAS) actives, as defined by the F.D.A.'s Tentative Final monograph on skin protectant drug products for over the counter human use (21 CFR §347). These monographed materials are known to provide multiple skin benefits, such as skin protection, itching relief, and irritation prevention. GRAS approved actives include, but are not limited to allantoin, zinc acetate, zinc oxide, salicylic acid, dimethicone, hyaluronic acid, calcium and salts thereof, dexpanthenol, protein hydrolystates, biabolol, glycyrrhetinic acid, vitamins A and D, tocopherols, and ascorbic acid or esters thereof. Examples of such ingredients include, but are not limited to: bisabolol and glycyrrhentic acid which are used as anti-inflammatories useful in the treatment of atopic dermatitis; Hyaluronic acid; butyrospermun parkii which has a moisturizing effect; and dimethicone whichis a protectant useful in the treatment of radiation dermatitis.

Therefore, in a preferred embodiment, the composition of the present invention will also contain one or more of the following: Hyaluronic acid, alpha-bisabolol, glycyrrhentic acid, allantoin, dexpanthenol, Lipsomal A, E, & C, and dimethicone. Each of these compounds is preferable in the composition at a concentration range of about 0.1% to 8% (% w/w), or more specifically 0.1% to 2.0% (% w/w).

According to a preferred embodiment, the composition of the invention will further contain one or more compounds with antioxidizing activity which may or may not be linked to liposomes. Examples of such compounds are ascorbic acid, tocopherols, bioflavonids, vitamin D, or nourishing liposomes with Vitamin A, C, E and/or astaxanthin. These liposomal systems are known to penetrate the upper layers of the skin and provide hydration while vitamins A, E, C, which can safely interact with free radicals, terminate the chain reaction before vital cell molecules are damaged.

In another embodiment, the present invention relates to a method of providing the composition as a transdermal base used for the percutaneous absorption of pharmaceutical and cosmaceutical active agent, including but not limited to coal tar, folic acid antagonist (such as methotrexate), antibiotics (such as erythromycin), antiemetic (such as scopolamine), a prostaglandin (such as misoprostol), an anti-inflammatory (such as ibuprofen, ketoprofen, or diclofenac), biologically active protein (such as phytospingosine or spingosine), analgesic (such as ketamine), a hormone (such as progesterone or testosterone, a cortosteroid (such as clobesterol), a vasodilator (such as isosorbide dinitrate), or other pharmaceutical agents.

In another embodiment, the present invention relates to a method of treating or repairing the barrier function of the skin. The composition of this embodiment provides benefits to the function of the skin barrier, which is important in treating dermatological conditions. Further, the composition of this embodiment is a film forming emulsion which can be used to provide an artificial hydrophobic barrier on the skin to treat skin conditions such as atopic dermatitis. A dysfunctional epidermal barrier is treated or prevented by topical application of a formulation comprised of certain major epidermal lipids. These combinations are effective both as moisturizing agents and as agents for the restoration of barrier function. It is generally accepted that the intercellular lamellar bilayer of the stratum corneum lipids are the key constituents in skin barrier functionality. The epidermal lipids consist of a mixture of polar and non-polar species which include, but are not limited to: ceramides, cholesterol, and fatty free acids. The listed weight of each of these species of lipids is ceramides (40%), cholesterol (20-25%), and fatty free acids (10-15%). Disorders of the skin (such as atopic dermatitis) that result from a dysfunctional epidermal barrier are treated or prevented by topical application of a formulation comprised of the three major epidermal lipids just discussed (ceramides, cholesterol, and fatty acids). As generally recognized by the art, combinations of these epidermal lipids in a molar concentration of 32:37:16:15 (cholesterol:cermaides:free fatty acids and minor fractions) are effective as moisturizers and as agents for restoration of barrier function.

A healthy stratum corneum should contain about 10% tightly held water, the presence of which is largely dependent on the presence of natural moisturizing factors, humectants, occlusion, and emollients. Each of these elements serves a function that helps to promote the overall health of the stratum corneum. In addition to providing occlusion, emollients fill the spaces between the corneocytes, which provides therapeutic improvements to defects in desquamation. Such emollients are thus able to smooth roughened skin, change the skin's appearance, lubricate, and replace natural skin lipids. Similar benefits are derived from the water attracting qualities of humectants. When applied to the skin in combination with silicones, humectants are able to attract water to the skin's surface while the silicones form a semi-occlusive barrier that promotes overall skin health by retaining moisture content. Moreover, current research shows that pH greatly influences the barrier nature of the stratum corneum. Particularly important to the functionality of the barrier created by the stratum corneum, is the selective permeability of the skin which is thought to be strongly related to the epidermal pH gradient. The pH gradient is likely to be generated from a number of sources and is also likely to control a number of processes essential for the formation of a competent epidermal permeability barrier.

This pH gradient has been suggested to activate enzymes responsible for the maintenance of the skin barrier function and to facilitate the desquamation process in the stratum corneum. In addition, the pH gradient significantly influences the permeation profile of ionizable drugs. Thus, elucidation of the mechanisms of the pH gradient formation and function should allow formulation of more effective approaches to ameliorating diseases caused or worsened by barrier dysfunction. This point is exemplified when one considers the impact of an abnormal pH gradient upon the ion states of the tissues. For example, calcium ions display a characteristic pH gradient in the epidermis where calcium levels increase from the basal layers to the granulosum layer and decline again in the stratum corneum. Thus, the presence of an abnormal pH gradient may impact the presence of calcium within the layers of skin. This impact can be construed as important as it has been suggested that the presence of calcium is required for the formation of intercellular lamellae which form when the content of lamellar bodies is reorganized.

According to a preferred embodiment, the composition of the invention will further contain dimethicone, a compound that temporarily protects injured or exposed skin or mucous membrane surfaces from harmful or annoying stimuli in addition to potentially providing relief to such surfaces. Dimethicone is FDA approved for over the counter human use as a skin protectant drug product. The preferred concentration range of dimethicone in the composition is about 0.5 to 10% (w/w %).

Polydimethylsiloxanes polymers or silicone gum have been shown to improve the substantivity of actives on the skin. These high molecular weight Polydimethylsiloxane polymers form invisible films on the skin which, although highly hydrophobic, are still permeable to moisture. Polydimethylsiloxane (PDMS) materials have unique properties directly driven by the mobility of the open Siloxane mesh, the low level of intermolecular interactions, and their low surface tension. As a result, PDMS polymers not only spread easily to form thin films over the skin and other organic substrates, they are also able to spread over their own absorbed film. In turn, this allows silicones to more readily wet, spread, and subsequently adhere to skin. Further, coupled with visco-elastic behavior, this property reinforces the silicone's ability to adhere to skin and subsequently release from skin allowing for the expected performance of PDMS as true pressure sensitive adhesives. Finally, PDMS is very permeable to the diffusion of various substances including active drugs. Each of these described properties is appropriate for transdermal and topical delivery applications.

Trimethylsiloxysiloxysilicate/dimethiconol crosspolymer, a unique Siloxane, has Theological properties similar to FDA-approved silicone pressure sensitive adhesives. Currently used in transdermal drug delivery systems, these Siloxane polymers possess film barrier properties, wash-off resistance, and durability (soft and pliable). Further, the high permeability of these Siloxane polymers to small molecules such as active ingredients also makes them very attractive for use as controlled-release vehicles. Similar benefits are derived from the ability of silicone polyethers (dimethicone copolyol) to form vesicles. By using dimethicone peg-12 to form these vesicles, both hydrophilic and hydrophobic actives can be separated from each other. The hydrophobic moiety of the surfactant is oriented toward the inside of the bilayer, while the hydrophilic moiety is facing the outside of the bilayer. This protection is obtained because hydrophobic actives can be distributed inside the bilayer, while hydrophilic actives will be in solution in the water phase. This protective mechanism further offers a way to reduce skin irritation caused by actives that are too irritating to be incorporated directly into a formula. Additionally, from an alternative standpoint, the aesthetics in these products should not be ignored because sensory characteristics are critical to patient compliance with treatment of chronic diseases. This emulsifier is beneficial because of its ability to emulsify a variety of oils and to form silicone vesicles, which advantageously combine aesthetic benefits with effective agent delivery.

Steroxytromethylsilane and steryl alcohol (silicone wax) also has aesthetic properties as a result of silicone moiety. The inclusion of this substance improves compatibility with organic ingredients and provides semi-occlusivity. In turn, silicone wax helps facilitate the formation of a very thin semi-occlusive film that adds lubricity to the skin.

According to a preferred embodiment the composition further contains one or more compounds which are natural fats (oils) or fatty acids. These compounds are present in the composition at a concentration of about 0.1% to about 10.0% w/w.

The following lists are for the purpose of elucidating the kinds of compounds that may be used, they are not meant to limit the present invention in any manner. Where natural fats (oils) are used, they may be selected from avocado oil, borage oil, coconut oil, oenothera oil, wheat germ oil, cotton seed oil, sunflower oil, olive oil, chamomile oil, palm oil, and sphingolipids. Where fatty acids are used, they may be selected from lauric acid, palmitic acid, stearic acid, linoleic acid, in addition to, saturated fatty acids, unsaturated fatty acids, or polyunsaturated fatty acids. Examples of saturated fatty acids include, but are not limited to: caprylic, capric, palmitic, steric, or arachidic. Examples of unsaturated fatty acids include, but are not limited to: palmitoleic acid, linoleic acid, alpha linolenic, or arachidonic acid. Examples of polyunsaturated fatty acids include, but are not limited to: eicosapentaenoic acid, docosahexaenoic acid, gamma-linolenic acid, or arachidonic acid and mixtures thereof.

According to a preferred embodiment, the composition of the invention will further contain one or more compounds wherein the sterol is selected from cholesterol, sitosterol, stigmasterol, ergosterol, cholesteryl acetate, Phytosterols, cholesterol NF, dihydrocholesterol, avocado sterols, and mixtures thereof.

The present invention also relates to a method of treating an inflammatory condition of the skin which consists of topically administering to the site of the condition a composition comprising:
Phase A: Dimethiconol (hydroxyl-terminated polydimethylsiloxane) (1.0-35.0% w/w), dimethicone-350 (Polydimethylsiloxane-350) (1-20.0% w/w);
Phase B: Cyclomethicone-5 nf (decamethylcyclopentasiloxane) (1.0-40.0% w/w);
Phase C: Alkymeth Siloxane copolyol-lauryl peg/ppg 18/18 methicone (alkymethyl Siloxane copolyol) (1.0-5.0% w/w);
Phase D: Cyclopentasiloxane and Dimethicone Crosspolymer (silicone elastomer and decamethylcyclopentasiloxane) (1.0-75.0% w/w), Stearoxytrimethylsilane and stearyl alcohol (silicone wax) (1.0-35.0%);
Phase E: sodium polyacrylate and dimethicone and cyclopentasiloxane and trideceth-6 and peg/ppg-18/18 dimethicone (sodium polyacrylate in dimethicone) (1.0-10.0% w/w), glycyrrhetinic acid (3% w/w), bisabolol (3.0% w/w), liposomes with vitamins A, E, C (0.5% w/w), hyaluronic acid(1.0% w/w), skin ceramide(Ceramide 3, Ceramide 6II, Ceramide 1, Phytosphingosine, Cholesterol), glycerin (1.0% w/w), butyrospermun parkii(1% w/w), and caprylic/ capric triglycerides (1.0% w/w); and
Phase F: Deionizer water (0.1-70% w/w).

Such composition, according to one embodiment, is prepared by a method the includes the steps of: (1) mixing ingredients of phase A together; (2) mixing ingredients of phase B together; (3) mixing ingredients of phase C together; (4) heating phase D to 50 degrees Celsius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; and (8) continuing mixing for 15 minutes and then homogenizing the emulsion.

In another embodiment, the present invention relates to a method indicated to manage and relieve the burning experienced with various types of dermatoses, including atopic dermatitis, allergic contact dermatitis, and radiation dermatitis. This method entails topically administering to the site of the condition, a composition comprising:
Phase A: Dimethiconol (hydroxyl-terminated polydimethylsiloxane) (2.0% w/w), dimethicone-350 (Polydimethylsiloxane-350) (5.0% w/w);
Phase B: Cyclomethicone-5 nf (decamethylcyclopentasiloxane) (10.0% w/w);
Phase C: Alkymeth Siloxane copolyol-lauryl peg/ppg 18/18 methicone (alkymethyl Siloxane copolyol) (2.0% w/w), Peg-12 Dimethicone (4.0% w/w), glycyrrhetinic acid (3% w/w), bisabolol (3.0% w/w), liposomes with vitamins A, E, C (0.5% w/w), hyaluronic acid(1.0% w/w), skin ceramide(Ceramide 3, Ceramide 6II, Ceramide 1, Phytosphingosine, Cholesterol), glycerin (1.0% w/w), butyrospermun parkii(1% w/w), and caprylic/ capric triglycerides (1.0% w/w).
Phase D: Cyclopentasiloxane and Dimethicone Crosspolymer ( silicone elastomer and decamethylcyclopentasiloxane) (10.0% w/w), Stearoxytrimethylsilane and stearyl alcohol (silicone wax) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (56.0% w/w) and preservative.

Such composition, according to one embodiment, is prepared by a method the includes the steps of: (1) mixing ingredients of phase A together; (2) mixing ingredients of phase B together; (3) mixing ingredients of phase C together; (4) heating phase D to 50 degrees Celsius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; and (8) continuing mixing for 15 minutes and then homogenizing the emulsion.

In a another embodiment, the present invention relates to a method of treating or preventing inflammatory conditions selected from dermatitis conditions and skin impairments such as atopic dermatitis, contact dermatitis, allergic contact dermatitis, allergic dermatitis, psoriasis pustulosa, psoriatic erythroderma, psoriasis arthropatica, rosacea, chronic actinic dermatitis, photouticaria, acne vulgaris, subacuta, chronica, juvenile and adult acne, papulous, pustulous, prurigo, acne nodose, acne conglobata, senile acne, acne tetrad, acne neonatorum, excoriated acneacne cosmetica, folliculitits, decubitis, ulus cruris, localized scratch dermatitis rhinophyma, perioral dermatitis, ichthyosis, xerosis, polymorphic photodermatosis, photodermatosis, radiation dermatitis, contact eczema, dyshidrosiform eczema, contact urticara, itching, age related wrinkles, sun damage, psoriasis vulgaris, flaking eczema, seborrheic dermatitis, nummular dermatitis, chronic dermatitis of hands and feet, generalized exfoliative dermatitis, neonatal dermatitis, pediatric dermatitis, scratch dermatitis, contact eczema, allergic contact eczema, photoallergic eczema, and diaper dermatitis. In preferred embodiments, this method of preventing inflammatory conditions of the skin entails topically administering to the site of the condition a composition comprising: This method entails topically administering to the site of the condition a composition comprising:
Phase A: Dimethiconol (hydroxyl-terminated polydimethylsiloxane) (2.0% w/w), dimethicone-350 (Polydimethylsiloxane-350) (5.0% w/w);
Phase B: Cyclomethicone-5 nf (decamethylcyclopentasiloxane) (10.0% w/w);
Phase C: Alkymeth Siloxane copolyol-lauryl peg/ppg 18/18 methicone (alkymethyl Siloxane copolyol) (2.0% w/w), glycyrrhetinic acid (3% w/w), bisabolol (3.0% w/w), liposomes with vitamins A, E, C (0.5% w/w), hyaluronic acid(1.0% w/w), skin ceramide(Ceramide 3, Ceramide 6II, Ceramide 1, Phytosphingosine, Cholesterol), glycerin (1.0% w/w), butyrospermun parkii(1% w/w), allantoin (0.5% w/w), and caprylic/ capric triglycerides (1.0% w/w).
Phase D: Cyclopentasiloxane and Dimethicone Crosspolymer ( silicone elastomer and decamethylcyclopentasiloxane) (10.0% w/w), Stearoxytrimethylsilane and stearyl alcohol (silicone wax) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (56.0% w/w) and preservative.

Such composition, according to one embodiment, is prepared by a method the includes the steps of: (1) mixing ingredients of phase A together; (2) mixing ingredients of phase B together; (3) mixing ingredients of phase C together; (4) heating phase D to 50 degrees Celsius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; and (8) continuing mixing for 15 minutes and then homogenizing the emulsion.

In another embodiment, the present invention relates to a method indicated to manage and relieve mucosal inflammatory conditions and neuropathic pain, particularly of the mouth and gingival. Traumatic neuropathies can occur following dental procedures such as, extractions, endodontic treatment and dental implant insertions which cause severe pain. The treatment for neuropathic pain is to topically administer the claimed composition in combination with anti-inflammatories such as ketoprofen or diclofenac, anticonvulsant such as gabapentin or carbamazepine and local anesthetic, such as lidocaine.

Optionally, in another embodiment, the present invention relates to a method used for the treatment of hypertrophic scars. Such method entails application of composition to the scar tissue. The composition is effective in reducing the size and appearance of scars. This beneficial response is due to barrier function repair, occlusivity, and skin hydration. This method entails topically administering to the site of the condition a composition that includes:
Phase A: Dimethiconol (hydroxyl-terminated polydimethylsiloxane) (2.0% w/w), dimethicone-350 (Polydimethylsiloxane-350) (5.0% w/w);
Phase B: Cyclomethicone-5 nf (decamethylcyclopentasiloxane) (10.0% w/w);
Phase C: Alkymeth Siloxane copolyol-lauryl peg/ppg 18/18 methicone (alkymethyl Siloxane copolyol) (2.0% w/w), glycyrrhetinic acid (3% w/w), bisabolol (3.0% w/w), liposomes with vitamins A, E, C (0.5% w/w), hyaluronic acid(1.0% w/w), skin ceramide(Ceramide 3, Ceramide 6II, Ceramide 1, Phytosphingosine, Cholesterol), glycerin (1.0% w/w), butyrospermun parkii(1% w/w), allantoin (0.5% w/w), and caprylic/capric triglycerides (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (56.0% w/w) and preservative.

Such composition, according to one embodiment, is prepared by a method the includes the steps of: (1) mixing ingredients of phase A together; (2) mixing ingredients of phase B together; (3) mixing ingredients of phase C together; (4) heating phase D to 50 degrees Celsius while mixing ingredients of phase D together; (5) incorporating Phases A, B, C, D together while mixing; (6) adding phase mixture (A, B, C, D) to Phase F while mixing; (7) adding Phase E to Phase mixture (A, B, C, D, F) while mixing to thicken; and (8) continuing mixing for 15 minutes and then homogenizing the emulsion.

It should be noted that the above composition may be used alone or in combination with one or more of the following active ingredients: glycyrrhetinic acid (3% w/w), bisabolol (3.0% w/w), liposomes with vitamins A, E, C (0.5% w/w), hyaluronic acid(1.0% w/w), skin ceramide(Ceramide 3, Ceramide 6II, Ceramide 1, Phytosphingosine, Cholesterol), glycerin (1.0% w/w), butyrospermun parkii(1% w/w), allantoin (0.5% w/w), and caprylic/capric triglycerides (1.0% w/w).

Compositions, in accordance with embodiments of the present invention, are applied topically and as frequently as required to achieve the desired therapeutic response. In other words, the compositions are applied in sufficient amounts to provide the desired effect without the undesirable side effects of the active or drug.

### EXAMPLES

The following series of examples are presented by way of illustration and not by way of limitation of the scope of the present invention.

### Example 1

The instant example describes a composition that is topically administered for the treatment of atopic dermatitis, allergic contact dermatitis, seborrheic dermatitis, radiodermatitis, xerosis, psoriasis, or atopia.
Phase A: Dimethiconol (hydroxyl-terminated polydimethylsiloxane) (4.0% w/w), dimethicone-350 (Polydimethylsiloxane-350) (5.0% w/w);
Phase B: Cyclomethicone-5 nf (decamethylcyclopentasiloxane) (15.0% w/w);
Phase C: Alkymeth Siloxane copolyol-lauryl peg/ppg 18/18 methicone (alkymethyl Siloxane copolyol) (2.0% w/w), Alpha bisabolol (1.5% w/w), glycyrrhizinic acid (2.0% w/w), liposomal with A, E, C (2.0% w/w), cholesterol (1.5% w/w), allantoin (0.5% w/w), caprylic/capric triglyceride (2.0% w/w), glycerin (0.5% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (42.87% w/w), kathon CG (0.05% w/w) and germaben II (0.08% w/w), all phases totaling 100%

### Example 2

The present example shows a formulation of the present invention used for treating and preventing inflammatory conditions and other skin impairments.
Phase A: Dimethiconol (hydroxyl-terminated polydimethylsiloxane) (2.0% w/w), dimethicone-350 (Polydimethylsiloxane-350) (5.0% w/w);
Phase B: Cyclomethicone-5 nf (decamethylcyclopentasiloxane) (15.0% w/w);
Phase C: Alkymeth Siloxane copolyol-lauryl peg/ppg 18/18 methicone (alkymethyl Siloxane copolyol) (2.0% w/w). Alpha bisabolol (1.5% w/w), glycyrrhizinic acid (2.0% w/w), liposomal with A, E, C (2.0% w/w), cholesterol (3.0% w/w), panthenol (0.2% w/w), shea blend emulsion (0.3% w/w), sulfate, caprylic/capric triglyceride (2.0% w/w), glycerin (0.5% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (43.37% w/w), kathon CG (0.05% w/w), and germaben II (0.08% w/w), all phases totaling 100%.

### Example 3

The present example describes a formulation used for treating or repairing the barrier function of the skin. During this treatment or repair the composition claimed provides benefits to the function of the skin barrier, an important part of treating dermatological conditions.
Phase A: Dimethiconol (hydroxyl-terminated polydimethylsiloxane) (5.0% w/w), dimethicone-350 (Polydimethylsiloxane-350) (8.0% w/w);
Phase B: Cyclomethicone-5 nf (decamethylcyclopentasiloxane) (12.0% w/w);
Phase C: Alkymeth Siloxane copolyol-lauryl peg/ppg 18/18 methicone (alkymethyl Siloxane copolyol) (2.0% w/w), Alpha bisabolol (1.5% w/w), linolenic acid (2.0% w/w), liposomal with A, E, C (2.0% w/w), cholesterol (2.0% w/w), borage oil (0.5% w/w), caprylic/capric triglyceride (2.0% w/w), zinc acetate (0.3% w/w), glycerin (0.5% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (41.70% w/w), kathon CG (0.05% w/w), and germaben II (0.08% w/w), all phases totaling 100%.

### Example 4

The present example describes a formulation for a transdermal base used for dental neuropathic pain.
Phase A: Dimethiconol (hydroxyl-terminated polydimethylsiloxane) (2.0% w/w), dimethicone-350 (Polydimethylsiloxane-350) (3.0% w/w);
Phase B: Cyclomethicone-5 nf (decamethylcyclopentasiloxane) (12.0% w/w);
Phase C: Alkymeth Siloxane copolyol-lauryl peg/ppg 18/18 methicone (alkymethyl Siloxane copolyol) (2.0% w/w), glycyrrhizinic acid (2.0% w/w), liposomal with A, E, C (2.0%), cholesterol (1.0% w/w), gabapentin (5.0% w/w), caprylic/capric triglyceride (0.5% w/w), glycerin (0.5% w/w), and sodium hyaluronate (1.0% w/w), peg-12 dimethicone (4.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (44.87% w/w), kathon CG (0.05% w/w), and germaben II (0.08% w/w), totaling 100%.

### Example 5

The present example describes a formulation of the base composition used for the treatment of hypertrophic and keloid scars.
Phase A: Dimethiconol (hydroxyl-terminated polydimethylsiloxane) (6.0% w/w), dimethicone-350 (Polydimethylsiloxane-350) (10.0% w/w);
Phase B: Cyclomethicone-5 nf (decamethylcyclopentasiloxane) (20.0% w/w);
Phase C: Alkymeth Siloxane copolyol-lauryl peg/ppg 18/18 methicone (alkymethyl Siloxane copolyol) (2.0% w/w), sodium hyluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (40.87% w/w), kathon CG (0.05% w/w), and germaben II (0.08% w/w); all phases totaling 100%.

### Example 6

The present example describes a formulation to be used as a barrier protectant for the skin by creating an occlusive film on the surface of the skin.
Phase A: Dimethiconol (hydroxyl-terminated polydimethylsiloxane) (6.0% w/w), dimethicone-350 (Polydimethylsiloxane-350) (10.0% w/w);
Phase B: Cyclomethicone-5 nf (decamethylcyclopentasiloxane) (20.0% w/w);
Phase C: Alkymeth Siloxane copolyol-lauryl peg/ppg 18/18 methicone (alkymethyl Siloxane copolyol) (2.0% w/w), glycyrrhizinic acid (2.0% w/w), Phytosterols (2.0% w/w), liposomal with A, E, C (2.0% w/w), cholesterol (3.0% w/w), sodium hyaluronate (1.0% w/w), bisabolol (5.0% w/w), caprylic/capric triglyceride (2.0% w/w), glycerin (0.5% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (24.37% w/w), kathon CG (0.05% w/w), and germaben II (0.08% w/w), all phases totaling 100%.

### Example 7

The present example describes a formulation used to provide moisturizing and occlusive effects that protects the skin.
Phase A: Dimethiconol (hydroxyl-terminated polydi methyl siloxane) (6.0% w/w), dimethicone-350 (Polydimethylsiloxane-350) (10.0% w/w);
Phase B: Cyclomethicone-5 nf (decamethylcyclopentasiloxane) (20.0% w/w);
Phase C: Alkymeth Siloxane copolyol-lauryl peg/ppg 18/18 methicone (alkymethyl Siloxane copolyol) (2.0% w/w), bisabolol (2.0% w/w), liposomal with A, E, C (2.0% w/w), cholesterol (3.0% w/w), zinc oxide (5.0% w/w), caprylic/capric triglyceride (2.0% w/w), glycerin (3.0% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (23.87% w/w), kathon CG (0.05% w/w), and germaben II (0.08% w/w), all phases totaling 100%.

### Example 8

The following describes preparation of nicotinamide 4%, a water soluable b-complex vitamin that occurs as a crystalline powder, in the claimed base composition. Nicotinamide 4% is free soluble in water and has a pka value of 0.5. The composition is useful as an anti-inflammatory for treatment of skin conditions such as acne vulgaris.
Phase A: Dimethiconol (hydroxyl-terminated polydimethylsiloxane) (6.0% w/w), dimethicone-350 (Polydimethylsiloxane-350) (10.0% w/w);
Phase B: Cyclomethicone-5 nf (decamethylcyclopentasiloxane) (20.0% w/w);
Phase C: Alkymeth Siloxane copolyol-lauryl peg/ppg 18/18 methicone (alkymethyl Siloxane copolyol) (2.0% w/w), niacinamide (4.0% w/w), glycyrrhizinic acid (2.0% w/w), liposomal with A, E, C (2.0% w/w), cholesterol (3.0% w/w), caprylic/capric triglyceride (2.0% w/w), glycerin (0.5% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (27.37% w/w), kathon CG (0.05% w/w), and germaben II (0.08% w/w), totaling 100%.

### Example 9

The present example describes a formulation used to manage and relieve the burning experience with various types of dermatosis.
Phase A: Dimethiconol (hydroxyl-terminated polydimethylsiloxane) (6.0% w/w), dimethicone-350 (Polydimethylsiloxane-350) (10.0% w/w);
Phase B: Cyclomethicone-5 nf (decamethylcyclopentasiloxane) (15.0% w/w);
Phase C: Alkymeth Siloxane copolyol-lauryl peg/ppg 18/18 methicone (alkymethyl Siloxane copolyol) (2.0% w/w), glycyrrhizinic acid (2.0% w/w), liposomal with A, E, C (2.0% w/w), cholesterol (3.0% w/w), bisabolol (2.0% w/w), caprylic/capric triglyceride (2.0% w/w), glycerin (1.0% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (33.87% w/w), kathon CG (0.05% w/w), and germaben II (0.08% w/w); all totaling 100%.

### Example 10

The present example describes a formulation of managing and relieving the burning experienced with various types of dermatoses including atopic dermatitis, allergic contact dermatitis, and radiation dermatitis.
Phase A: Dimethiconol (hydroxyl-terminated polydimethylsiloxane) (6.0% w/w), dimethicone-350 (Polydimethylsiloxane-350) (10.0% w/w);
Phase B: Cyclomethicone-5 nf (decamethylcyclopentasiloxane) (15.0% w/w);
Phase C: Alkymeth Siloxane copolyol-lauryl peg/ppg 18/18 methicone (alkymethyl Siloxane copolyol) (2.0% w/w), glycyrrhizinic acid (2.0% w/w), liposomal with A, E, C (2.0% w/w), cholesterol (3.0% w/w), bisabolol (2.0% w/w), caprylic/capric triglyceride (2.0% w/w), glycerin (1.0% w/w), and sodium hyaluronate (1.0% w/w);
Phase D: Cyclopentasiloxane and Dimethicone Crosspolymer (silicone elastomer and decamethylcyclopentasiloxane) (10.0% w/w), Stearoxytrimethylsilane and stearyl alcohol (silicone wax ) (5.0% w/w);
Phase E: sodium polyacrylate and dimethicone and cyclopentasiloxane and trideceth-6 and peg/ppg-18/18 dimethicone (sodium polyacrylate in dimethicone) (5.0% w/w); and
Phase F: Deionizer water (33.87% w/w), kathon CG (0.05% w/w) and germaben II (0.08% w/w), all phases totaling 100% .

The present invention, which relates to a composition useful as a vehicle for percutaneous absorption of pharmaceutical and cosmaceutical active agents, has been described. Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments, and it is intended that the appended claims cover any and all such applications, modifications, and embodiments within the scope of the present invention.

The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically.

What is claimed is:

1. A composition for the delivery of pharmaceutically-active substances for the treatment of inflammatory dermatosis and other pathological conditions of the skin, the composition comprising:
Phase A: dimethiconol(hydroxyl-terminated polydimethylsiloxane) (1.0-35.0% w/w), dimethicone-350 (Polydimethylsiloxane-350) (1.0-20.0% w/w);
Phase B: Cyclomethicone-5 nf (decamethylcyclopentasiloxane) (1.0-40.0% w/w);
Phase C: Alkymeth Siloxane copolyol-lauryl peg/ppg 18/18 methicone (alkymethyl Siloxane copolyol) (1.0-5.0% w/w);
Phase D: Cyclopentasiloxane and Dimethicone Crosspolymer (silicone elastomer and decamethylcyclopentasiloxane) (1.0-75.0% w/w), Stearoxytrimethylsilane and stearyl alcohol (silicone wax) (1.0-35.0% w/w);
Phase E: Sodium polyacrylate and dimethicone and cyclopentasiloxane and trideceth-6 and peg/ppg-18/18 dimethicone (sodium polyacrylate in dimethicone) (1.0-10.0% w/w); and
Phase F: Deionizer water (0.1-70.0% w/w).

2. The composition according to claim 1, further comprising peg-12 dimethicone as an emulsifier for various oils, sterol and phospolipids.

3. The composition according to claim 1, further comprising at least one of Kathon CG and Germaben II.

4. A method of treating a hypertrophic scar, the method comprising: applying onto a hypertrophic scar a composition that includes:
Phase A: dimethiconol(hydroxyl-terminated polydimethylsiloxane) (1.0-35.0% w/w), dimethicone-350 (Polydimethylsiloxane-350) (1.0-20.0% w/w);
Phase B: Cyclomethicone-5 nf (decamethylcyclopentasiloxane) (1.0-40.0% w/w),
Phase C: Alkymeth Siloxane copolyol-lauryl peg/ppg 18/18 methicone (alkymethyl Siloxane copolyol) (1.0-5.0% w/w);
Phase D: Cyclopentasiloxane and Dimethicone Crosspolymer (silicone elastomer and decamethylcyclopentasiloxane) (1.0-75.0% w/w), Stearoxytrimethylsilane and stearyl alcohol (silicone wax) (1.0-35.0% w/w);
Phase E: Sodium polyacrylate and dimethicone and cyclopentasiloxane and trideceth-6 and peg/ppg˜18/18 dimethicone (sodium polyacrylate in dimethicone) (1.0-10.0% w/w); and
Phase F: Deionizer water (0.1-70.0% w/w), having contained therein a dermatologically effective amount of an active ingredient capable of reducing the size of the scar or improving the appearance.

5. The composition according to claim 1, wherein the composition is in the form of one of oil/water emulsion, water/oil emulsion, water/oil/water emulsion, silicone/water emulsion, water/silicone emulsion, water/wax emulsion, oil/water/silicone emulsion hydro-alcohol gel, cream, and ointment.

6. The composition according to claim 1, further comprising at least one of glycyrrhizic acid, hydrolyzed hyaluronic acid, bisabolol, Ceramide 3, Ceramide 6II, Ceramide 1, Phytosphingosine, behenic acid. Cholesterol, liposomes with vitamins A, E, C, dexapanthenol, niacinamide, and glycerin.

7. The composition according to claim 1, further comprising at least one compound selected from the group consisting of: avocado oil, borage oil, coconut oil, oenothera oil, wheat germ oil, cotton seed oil, sunflower oil, olive oil, chamomile oil, palm oil, and sphingolipids, lauric acid, palmitic acid, stearic acid, linoleic acid, capric/caprylic triglycerides, palmitic, steric, palmitoleic acid, linoleic acid, alpha linolenic, or arachidonic acid, eicosapentaenoic acid, docosahexaenoic acid, gamma-linolenic acid, and arachidonic acid.

8. The composition according to claim 1, wherein the composition further includes one or more of allantoin, zinic oxide, glycerin, and zinc acetate.

9. The composition according to claim 1, wherein the composition further includes one or more of sterol, sterol esters, cholesterol, protein hydrolystates, cholesterol acetate, cholesteryl sitosterol, phytosterol, ceramide np, ceramide ns, ceramide eo, ceramides eop, phytosphingosine, and cholesteryl isosterate.

10. The composition according to claim 1, wherein the composition further includes one or more compounds with antioxidizing activity.

11. A composition suitable for topical administration used for the treatment of inflammatory conditions, dermatoses, and skin impairments the composition comprising;
Phase A: Dimethiconol (hydroxyl-terminated polydimethylsiloxane) (3.0% w/w), dimethicone-350 (Polydimethylsiloxane-350) (3.0% w/w);
Phase B: Cyclomethicone-5 nf (decamethylcyclopentasiloxane) (10.0% w/w);
Phase C: Alkymeth Siloxane copolyol-lauryl peg/ppg 18/18 methicone (alkymethyl Siloxane copolyol) (2.0% w/w), Alpha hisaboiol (1.5% w/w), glycyrrhizinic acid (1.5% w/w), hydrolized hyaluronic acid (1.0% w/w),skin ceramide(Ceramide 3, Ceramide 6II, Ceramide 1, Phytosphingosine, Cholesterol) (1% w/w), liposomal with vitamins A, E, C (1.0% w/w), allantoin (0.5% w/w), caprylic/capric triglyceride (1.0% w/w), butyrospermun parkii(1% w/w), glycerin (1.0% w/w);
Phase D: Cyclopentasiloxane and Dimethicone Crosspolymer (silicone elastomer and decamethylcyclopentasiloxane) (8.0% w/w), Stearoxytrimethylsilane and stearyl alcohol (silicone wax) (3.0% w/w);
Phase E: sodium polyacrylate and dimethicone and cyclopentasiloxane and trideceth-6 and peg/ppg-18/18 dimethicone (sodium polyacrylate in dimethicone), (5.0% w/w); and
Phase F: Deionizer water (56.37% w/w), kathon CG (0.05% w/w), and germaben II (0.08% w/w); totaling 100% w/w.

12. A method for treating atopic dermatitis, allergic contact dermatitis, seborrheic dermatitis, radiation dermatitis, xerosis, psoriasis, or atopia comprising topically administering a composition comprising:
Phase A: Dimethiconol (hydroxyl-terminated polydimethylsiloxane), dimethicone-350 (Polydimethylsiloxane-350);
Phase B; Cyclomethicone-5 nf (decamethylcyclopentasiloxane);
Phase C: Alkymeth Siloxane copolyol-lauryl peg/ppg 18/18 methicone (alkymethyl Siloxane copolyol), Alpha, bisabolol, glycyrrhizinic acid, hydrolized hyaluronic acid, skin ceramide(Ceramide 3, Ceramide 6II, Ceramide I, Phytosphingosine, Cholesterol), vitamins A, E, C, allantoin, caprylic/capric triglyceride, butyrospermun parkii, glycerin;
Phase D: Cyclopentasiloxane and Dimethicone Crosspolymer (silicone elastomer and decamethylcyclopentasiloxane), Stearoxytrimethylsilane and stearyl alcohol (silicone wax;
Phase E: sodium polyacrylate and dimethicone and cyclopentasiloxane and trideceth-6 and peg/ppg-18/18 dimethicone (sodium polyacrylate in dimethicone); and
Phase F: Deionizer water, kathon CG and germaben II, to a subject in need of such treatment in an amount effective to treat dermatitis, allergic contact dermatitis, seborrheic dermatitis, radiation dermatitis, xerosis, psoriasis, or atopia.

13. The method according to claim 12, to manage and relieve the burning experienced with various types of dermatoses including, but by no means limited to atopic dermatitis, allergic contact dermatitis, and radiation dermatitis, comprising of topically applying therapeutically effective amount of the composition to the skin.

14. The method according to claim 12, for treating or enhancing the barrier function of the epidermis of the user, comprising of topically applying therapeutically effective amount of the composition to the epidermis.

15. The method according to claim 12, wherein the inflammatory condition is selected from the group consisting of dermatoses, dermatitis conditions and pathological skin impairments including, but not limited to: atopic dermatitis, contact dermatitis, allergic contact dermatitis, allergic dermatitis, psoriasis pustulosa, psoriatic erythroderma, psoriasis arthropatica, rosacea, chronic actinic dermatitis, photouticaria, acne vulgaris, subacuta, chronica, juvenile and adult acne, papulous, pustulous prurigo, acne nodose, acne conglobata, senile acne, acne tetrad, acne neonatorum, excoriated acne, acne cosmetica, folliculitits, decubitis, ulus cruris, localized scratch dermatitis rhinophyma, perioral dermatitis, ichthyosis, xerosis, polymorphic photodermatosis, photodermatosis, radiation dermatitis, contact eczema, dyshidrosiform eczema, contact urticara, itching, age related wrinkles, sun damage, psoriasis vulgaris, flaking exzema, seborrheic dermatitis, rynauds syndrome, nummular dermatitis, chronic dermatitis of hands and feet, generalized exfoliative dermatitis, neonatal dermatitis, pediatric dermatitis, scratch dermatitis, contact eczema, allergic contact eczema, photoallergy eczema, and diaper dermatitis.
